(19) United States
(12) Patent Application Publication
(10) Pub. No.: US 2013/0253450 A1
(43) Pub. Date: Sep. 26, 2013

(54) SYSTEM AND METHOD FOR THE TREATMENT OF LICE WITH A LIQUID SOLUTION

(71) Applicant: Laura Marie Shelton, Morristown, NJ (US)

(72) Inventor: Laura Marie Shelton, Morristown, NJ (US)

(21) Appl. No.: 13/795,216

(22) Filed: Mar. 12, 2013

Related U.S. Application Data
(60) Provisional application No. 61/613,688, filed on Mar. 21, 2012.

(51) Int. Cl. A45D 19/04; A61M 35/00

(52) U.S. Cl. CPC A45D 19/04; A61M 35/00
USPC 604/290; 604/289

(57) ABSTRACT

The present invention relates generally to the treatment and eradication of lice. More particularly, the present invention relates to a system and method for treatment and eradication of lice through the submersion of an infected area in a liquid solution.

### CROSS-REFERENCE TO RELATED APPLICATIONS

This application claims the benefit of the following provisional applications, each of which is hereby incorporated by reference in its entirety: U.S. Pat. App. No. 61/613,688 filed on Mar. 21, 2012 and entitled “SYSTEM AND METHOD FOR THE TREATMENT OF LICE WITH A LIQUID SOLUTION.”

### FIELD OF THE INVENTION

The present invention relates generally to the treatment and eradication of lice. More particularly, the present invention relates to a system and method for treatment and eradication of lice through the submersion of an infected area in a liquid solution.

### BACKGROUND

About 6-12 million children ages 3-11 get infested with head lice in the U.S. each year. While lice do not cause disease and are not dangerous, they do cause considerable itching. Lice are notoriously difficult to treat. Pesticides have been the first line of treatment for years. However, many lice are now resistant to these products making an infestation all the more challenging.

In addition to the increased resistance of lice to these pesticides, many caregivers today are hesitant to use these treatments on their child's hair because they are concerned about potential side effects. These caregivers have resorted to pouring olive oil or mayonnaise on their child's hair and covering the head with plastic wrap overnight in an effort to suffocate the lice. This can be a very messy and inefficient process.

Another key to treating a lice infestation is nit picking Nits are lice eggs which adhere to a strand of hair. Caregivers are instructed to comb through their child's hair and pull these off one by one. This process can take several hours depending on the child's hair length and thickness and the degree of infestation.

Current methods of head lice removal or eradication require a child to be seated with a towel wrapped around his/her shoulders while a caregiver applies a product to either kill or suffocate the head lice. The application of these products is very painstaking since the caregiver must be certain to thoroughly apply product throughout the hair. The caregiver must section off the hair with a comb section by section. This process can easily take over an hour and it is quite difficult to apply the product to every section of the head. Children hate this process since it is so tedious.

After a period of approximately ten minutes, the child is instructed to either shampoo their hair (when treated with insecticides) or the caregiver is instructed to inspect and comb through the hair to remove lice and nits.

The tediousness and difficulty of treating head lice makes parents dread a note from school informing them of a lice outbreak in their child's classroom.

Therefore, there is a need in the art for a system and method for efficiently and effectively treating lice infestations without the use of pesticides. These and other features and advantages of the present invention will be explained and will become obvious to one skilled in the art through the summary of the invention that follows.

### SUMMARY OF THE INVENTION

Accordingly, it is an aspect of the present invention to provide a system and method for efficiently and effectively treating lice infestations without the use of pesticides.

According to an embodiment of the present invention, a system for the treatment of lice includes: a basin comprising a walled structure with a concave cavity configured to receive a lice treatment solution, wherein said lice treatment solution contains at least 95% ethyl alcohol.

According to an embodiment of the present invention, the walled structure of said basin further comprises a channel on one side configured to receive the neck of an individual to be treated.

According to an embodiment of the present invention, the lice treatment solution contains at least 0.05% bittering agent.

According to an embodiment of the present invention, the ethyl alcohol is at least 80 proof.

According to an embodiment of the present invention, the ethyl alcohol is 180 proof.

According to an embodiment of the present invention, the basin further comprises a timer.

According to an embodiment of the present invention, the timer is configured to produce a noticeable alert upon completion of a lice treatment.

According to an embodiment of the present invention, the timer is integrated into said walled structure of said basin.

According to an embodiment of the present invention, the basin further comprises a back rest portion configured to allow an individual to relax during receipt of a lice treatment session.

According to an embodiment of the present invention, a method for the treatment of lice includes the steps of: filling a basin comprising a walled structure with a concave cavity with a lice treatment solution to a level appropriate for entirely engulfing all hair and scalp of an individual; submerging said hair and scalp of said individual into said basin for a period of not less than five minutes; and removing said hair and scalp of said individual from said basin.

According to an embodiment of the present invention, a method for the treatment of lice includes the steps of: filling a basin comprising a walled structure with a timer and a concave cavity with a lice treatment solution to a level appropriate for entirely engulfing all hair and scalp of an individual; submerging said hair and scalp of said individual into said basin; engaging said timer; waiting for said timer to produce a perceptible notification; and removing said hair and scalp of said individual from said basin.

According to an embodiment of the present invention, the method includes the step of resting shoulders of the individual on said back rest portion during said submerging step.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

Referring to the drawings, wherein like reference numerals are used to designate like parts:

FIG. 1 shows a top view of a wash basin for use in the eradication of lice, in accordance with an embodiment of the present invention.

FIG. 2 shows a perspective view of a wash basin for use in the eradication of lice, in accordance with an embodiment of the present invention.

FIG. 3 shows a perspective view of a wash basin for use in the eradication of lice in use, in accordance with an embodiment of the present invention.

### DETAILED SPECIFICATION

The present invention relates generally to the treatment and eradication of lice. More particularly, the present invention relates to a system and method for treatment and eradication of lice through the submersion of the infected area in a liquid solution.

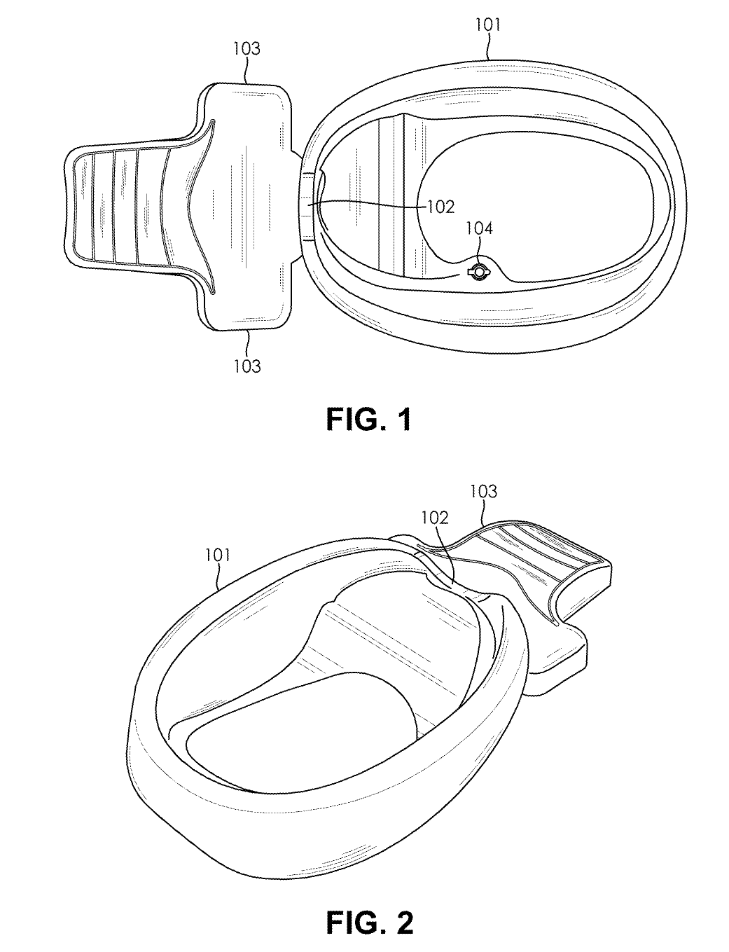

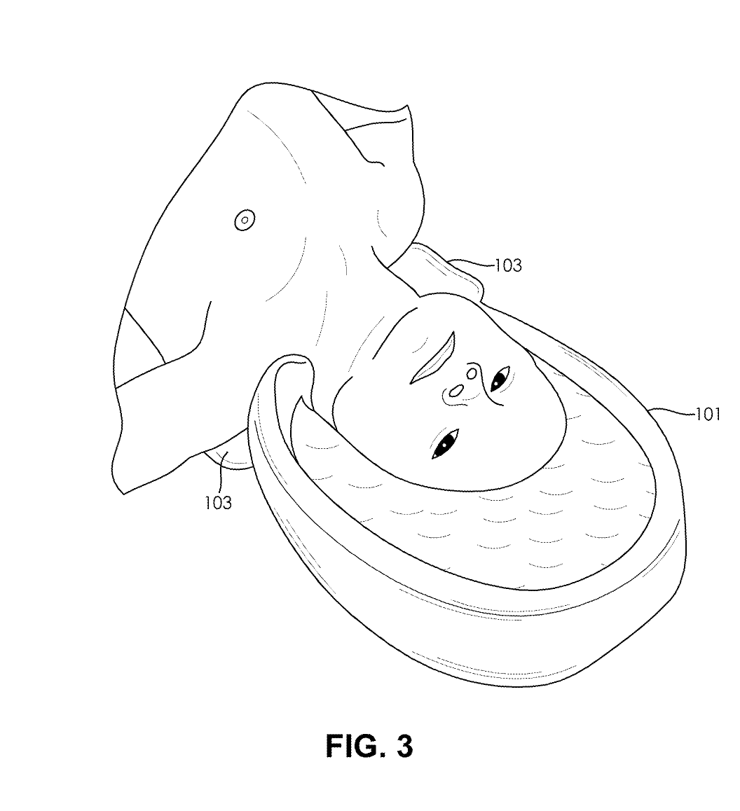

According to an embodiment of the present invention, a liquid solution is poured into or otherwise inserted into a basin capable of receiving a lice infected area of an individual's body. Most commonly, the lice infected area is the head and the basin is of a shape and structure not unlike wash basins found in salons. The basin should be of a width, length and depth capable of receiving the entirety of the lice infected area, preferably including the scalp of the affected individual. FIGS. 1-3 show exemplary embodiments of a basin in accordance with an embodiment of the present invention.

With respect to FIGS. 1-3, views of a preferred embodiment of the present invention are shown. In these figures, a basin is shown where a walled structure 101 forms a cavity for the retention of a lice treatment solution. The walled structure may have a channel 102, or lowered portion on one side for receipt of an individual's neck which will rest on the channel during the treatment. Optionally, the basin may have a back rest portion 103, configured to receive an individual's back and/or shoulders during treatment. The back rest portion 103 is provided to increase the comfort of an individual receiving treatment during a treatment session. In certain embodiments, the basin may be further comprised of a drain plug 104.

According to an embodiment of the present invention, the basin may be capable of being arranged in such a fashion as to allow the individual to lie prone or in a reclined position. In this manner, the individual receiving treatment may be provided comfort or relaxation during the procedure. An example of this is similar to the reclining seats attached to wash basins for washing hair found in salons. Allowing the individual to lie prone or otherwise relax during the procedure provides comfort to the individual and increases the likelihood that the individual will remain still for the duration of the treatment.

According to an embodiment of the present invention, the basin is filled with the liquid solution to a predetermined level or to a level which allows for the complete submersion of the infected area. Once filled to the desired level, the individual receiving treatment will submerge the infected area in the liquid solution. The individual will keep the infected area submerged until such time that all of the lice in the infected area have been eradicated. The process takes approximately ten minutes, but longer or shorter durations may be utilized. In certain embodiments of the present invention, the basin may include a timer set to provide an audible tone or other noticeable stimuli to identify to the individual or other person that the process is complete. The timer may be configured to be set to any desired time length, whether ten minutes or a longer or shorter period of time.

After the expiration of the submersion period, the lice in the infected area should all have been suffocated or otherwise eradicated. In addition, all the nits should all have been sufficiently dried out so as to be unable to hatch. The individual may then wash the infected area as usual (e.g., with a standard shampoo or other soap). At this point, the infected area should be clear of lice infestation.

According to an embodiment of the present invention, the liquid solution may be comprised of a denatured alcohol solution. Such a denatured alcohol solution may be comprised of an 80 proof or greater ethyl alcohol and a bittering agent. The ethyl alcohol works to kill and suffocate the lice and render the nits unable to hatch while the bittering agent allows for the denatured alcohol solution to be non-potable. The bittering agent also works to allow the denatured alcohol solution to avoid taxes based on potable alcohols.

According to an embodiment of the present invention, the denatured alcohol would be 80 proof or greater. One of ordinary skill in the art would appreciate that numerous 80 proof or greater ethyl alcohol may be utilized with embodiments of the present invention, and embodiments of the present invention are contemplated for use with any 80 proof or greater ethyl alcohol.

According to an embodiment of the present invention, the bittering agent may be one or more of denatonium benzoate, methanol, naphtha or pyridine. One of ordinary skill in the art would appreciate that there are numerous bittering agents that may be utilized with embodiments of the present invention, and embodiments of the present invention are contemplated for use with any bittering agent.

According to an embodiment of the present invention, the denatured ethyl alcohol solution may be comprised of 99%+80 proof or greater ethyl alcohol and less than 1% bittering agent. In a preferred embodiment, the denatured ethyl alcohol solution may be comprised of 99% 180 proof alcohol and less than 1% bittering agent.

In a preferred embodiment, using a denatured ethyl alcohol solution comprising 180 proof alcohol, a treatment comprises submerging the hair and scalp of an individual in the denatured ethyl alcohol solution for approximately 5 minutes. While 5 minutes is sufficient to provide the desired results, longer periods may be used to ensure complete eradication of infestations.

According to an embodiment of the present invention, the denatured alcohol solution described herein could be utilized without the use of a bittering agent. In this manner, the solution would be comprised of an 80 proof or greater ethyl alcohol. In this form, the denatured alcohol solution may be subject to additional taxes and increased risk that individuals could consume the denatured alcohol solution. However, the functionality of the denatured alcohol solution as it related to the eradication of lice would be unimpeded.

According to an embodiment of the present invention, the liquid solution could be comprised of a variety of liquids in various concentrations. One would appreciate that there are numerous liquid solutions that could be utilized to treat lice in accordance with embodiments of the present invention, and embodiments of the present invention are contemplated for use with any liquid solution appropriate for treating lice.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A system for the treatment of lice, said system comprising:
a basin comprising a walled structure with a concave cavity configured to receive a lice treatment solution, wherein said lice treatment solution contains at least 95% ethyl alcohol.

2. The system of claim 1, wherein said walled structure of said basin further comprises a channel on one side configured to receive the neck of an individual to be treated.

3. The system of claim 1, wherein said lice treatment solution contains at least 0.05% bittering agent.

4. The system of claim 1, wherein said ethyl alcohol is at least 80 proof.

5. The system of claim 1, wherein said ethyl alcohol is 180 proof.

6. The system of claim 1, wherein said basin further comprises a timer.

7. The system of claim 6, wherein said timer is configured to produce a noticeable alert upon completion of a lice treatment.

8. The system of claim 6, wherein said timer is integrated into said walled structure of said basin.

9. The system of claim 1, wherein said basin further comprises a back rest portion configured to allow an individual to relax during receipt of a lice treatment session.

10. A method for the treatment of lice, said method comprising the steps of:
filling a basin comprising a walled structure with a concave cavity with a lice treatment solution to a level appropriate for entirely engulfing all hair and scalp of an individual;
submerging said hair and scalp of said individual into said basin for a period of not less than five minutes; and
removing said hair and scalp of said individual from said basin.

11. The method of claim 10, wherein said lice treatment solution contains at least 0.05% bittering agent.

12. The method of claim 10, wherein said ethyl alcohol is at least 80 proof.

13. The method of claim 10, wherein said ethyl alcohol is 180 proof.

14. A method for the treatment of lice, said method comprising the steps of:
filling a basin comprising a walled structure with a timer and a concave cavity with a lice treatment solution to a level appropriate for entirely engulfing all hair and scalp of an individual;
submerging said hair and scalp of said individual into said basin;
engaging said timer;
waiting for said timer to produce a perceptible notification; and
removing said hair and scalp of said individual from said basin.

15. The method of claim 14, wherein said lice treatment solution contains at least 0.05% bittering agent.

16. The method of claim 14, wherein said ethyl alcohol is at least 80 proof.

17. The method of claim 14, wherein said ethyl alcohol is 80 proof.

18. The method of claim 14, wherein said basin further comprises a back rest portion.

19. The method of claim 18, further comprising the step of resting shoulders of the individual on said back rest portion during said submerging step.
